(19) United States
(12) Patent Application Publication
(10) Pub. No.: US 2021/0407630 A1
(43) Pub. Date: Dec. 30, 2021

(54) Clinical Trial Protocol Portal

(71) Applicant: Syneos Health, LLC, Morrisville, NC (US)

(72) Inventors: Wendy BALTER, Morrisville, NC (US); Michael COLLINSON, Morrisville, NC (US); Nick WILLHOFT, Morrisville, NC (US)

(73) Assignee: Syneos Health, LLC, Morrisville, NC (US)

(21) Appl. No.: 17/364,050

(22) Filed: Jun. 30, 2021

Related U.S. Application Data
(60) Provisional application No. 63/045,956, filed on Jun. 30, 2020.

(51) Int. Cl. G16H 10/20; G16H 80/00

(52) U.S. Cl. CPC G16H 10/20; G16H 80/00

(57) ABSTRACT

A system and a computer-implemented method for generating a protocol portal. A computing system receives a request to generate a protocol portal from a client device. The computing system generates a protocol portal for the client. The protocol portal includes one or more protocol portal templates. The computing system receives content for a protocol associated with the protocol portal from the client device. The computing system transforms the received content into a format compatible with the protocol portal. The computing system dynamically tags content uploaded by the client device. The computing system generates one or more graphical user interfaces for the protocol portal using the received content and the protocol portal templates.

### CROSS-REFERENCE TO RELATED APPLICATIONS

This application claims priority to U.S. Provisional Application Ser. No. 63/045,956, filed Jun. 30, 2020, which is hereby incorporated by reference in its entirety.

### FIELD OF DISCLOSURE

The present disclosure generally relates to a system and a method for generating a protocol portal.

### BACKGROUND

A protocol may define various content that define the objects or design of a clinical trial. When a protocol is initiated, end users are required to undergo a training process regarding the objects and designs of the clinical trial. Such process, conventionally, is time consuming and clients are sometimes unable to track end user compliance with the training procedure.

### SUMMARY

In some embodiments, a computer-implemented method for generating a protocol portal is disclosed herein. A computing system receives a request to generate a protocol portal from a client device. The computing system generates a protocol portal for the client device. The protocol portal includes one or more protocol portal templates. The computing system receives content for a protocol associated with the protocol portal from the client device. The computing system transforms the received content into a format compatible with the protocol portal. The computing system dynamically tags content uploaded by the client device. The computing system generates one or more graphical user interfaces for the protocol portal using the received content and the protocol portal templates.

In some embodiments, a non-transitory computer-readable medium is disclosed herein. The non-transitory computer readable medium includes one or more instructions that, when executed by a computing system, causes the computing system to perform operations. The operations include receiving, by the computing system, a request to generate a protocol portal from a client device. The operations further include generating, by the computing system, a protocol portal for the client. The protocol portal includes one or more protocol portal templates. The operations further include receiving, by the computing system, content for a protocol associated with the protocol portal from the client device. The operations further include transforming, by the computing system, the received content into a format compatible with the protocol portal. The operations further include dynamically tagging, by the computing system, content uploaded by the client device. The operations further include generating, by the computing system, one or more graphical user interfaces for the protocol portal using the received content and the protocol portal templates.

In some embodiments, a system is disclosed herein. The system includes a processor and a memory. The memory has programming instructions stored thereon, which, when executed by the processor, causes the system to perform operations. The operations include receiving a request to generate a protocol portal from a client device. The operations further include generating a protocol portal for the client, the protocol portal comprising one or more protocol portal templates. The operations further include receiving content for a protocol associated with the protocol portal from the client device. The operations further include transforming the received content into a format compatible with the protocol portal. The operations further include dynamically tagging content uploaded by the client device. The operations further include generating one or more graphical user interfaces for the protocol portal using the received content and the protocol portal templates.

### BRIEF DESCRIPTION OF THE DRAWINGS

So that the manner in which the above recited features of the present disclosure can be understood in detail, a more particular description of the disclosure, briefly summarized above, may be had by reference to embodiments, some of which are illustrated in the appended drawings. It is to be noted, however, that the appended drawings illustrate only typical embodiments of this disclosure and are therefore not to be considered limiting of its scope, for the disclosure may admit to other equally effective embodiments.

FIG. 1 is a block diagram illustrating an exemplary computing environment, according example embodiments.

FIG. 2 illustrates an example view of a graphical user interface presenting a protocol portal, according to example embodiments.

FIG. 3 illustrates an example view of a graphical user interface presenting a video synopsis, according to example embodiments.

FIG. 4 illustrates an example view of a graphical user interface presenting a resources hub, according to example embodiments.

FIG. 5A illustrates an example view of a graphical user interface presenting an updates page, according to example embodiments.

FIG. 5B illustrates an example view of a graphical user interface presenting an updates page, according to example embodiments.

FIG. 5C illustrates an example view of a graphical user interface presenting an updates page, according to example embodiments.

FIG. 5D illustrates an example view of a graphical user interface presenting an updates page, according to example embodiments.

FIGS. 6A-6C illustrates an example view of a searching portion of a graphical user interface, according to example embodiments.

FIG. 7 is a flow diagram illustrating a method of generating a protocol portal, according to example embodiments.

FIG. 8 is a flow diagram illustrating a method of generating search results, according to example embodiments.

FIG. 9A illustrates an example view of a graphical user interface through which a user can upload content for a protocol, according to example embodiments.

FIG. 9B illustrates an example view of a graphical user interface through which a client can upload content for a protocol, according to example embodiments.

FIG. 9C is a continuation of the graphical user interface of FIG. 9B through which a client can upload content for a protocol, according to example embodiments.

FIG. 9D illustrates an example view of a graphical user interface through which a client can update a protocol, according to example embodiments.

FIG. 9E is a continuation of the graphical user interface of FIG. 9D through which a client can upload content for a protocol, according to example embodiments.

FIG. 9F is a continuation of the graphical user interface of FIG. 9D through which a client can upload content for a protocol, according to example embodiments.

FIG. 9G is a continuation of the graphical user interface of FIG. 9D through which a client can upload content for a protocol, according to example embodiments.

FIG. 10A illustrates an example system configuration for implementing various embodiments of the present technology, according to example embodiments.

FIG. 10B illustrates an example system configuration for implementing various embodiments of the present technology, according to example embodiments.

FIG. 11 illustrates an example view of a graphical user interface through which a user can upload content for a formulary dossier, according to example embodiments.

FIG. 12 illustrates an example view of a graphical user interface options to customize a website for a given protocol, according to example embodiments.

FIG. 13 illustrates an example view of a graphical user interface providing a metrics dashboard, according to example embodiments.

To facilitate understanding, identical reference numerals have been used, where possible, to designate identical elements that are common to the figures. It is contemplated that elements disclosed in one embodiment may be beneficially utilized on other embodiments without specific recitation.

### DETAILED DESCRIPTION

One or more techniques described herein are generally directed to a system and method for generating a protocol portal. A protocol may define various content that define the objects or design of a clinical trial. For example, a protocol may include documentation regarding how a clinical trial will be conducted (e.g., the objective(s), design, methodology, statistical considerations, and organization of a clinical trial) and ensures the safety of the trial subjects and integrity of the data collected. Conventionally, investigator meetings are the first step in a clinical trial process. However, too much reliance is placed on the investigator meetings to train sites on protocols. Few site staff attend the meetings and those who do are often not responsible for day to day implementation of trials. Moreover, the dynamic of each trial creates a learning curve regarding interpretation and implementation of the protocol in addition to protocol amendments. This dynamic environment is not adequately addressed by currently available support items. Currently support materials tend to arrive several weeks after the investigator meeting and remain static through the life of the trial.

The one or more techniques described herein improve upon conventional processes by providing a system capable of generating protocol portals, which allow for easier user compliance, as well as a platform that can be easily updated throughout the life of the trial. Further, the one or more techniques described herein allow end users to search, not only within a protocol document (e.g., a PDF document), but also across any content associated with a protocol. This allows end users to search across documents, videos, audio clips, images, and the like.

The present system may act as a resource to support implementation of the protocol, reduce the number of protocol violations, and ultimately lead to speedier study completion. The more investigators understand the study, the fewer unanswered questions they will have. Ultimately, this may lead to fewer protocol violations, and improvement in other metrics that are important to study sponsors.

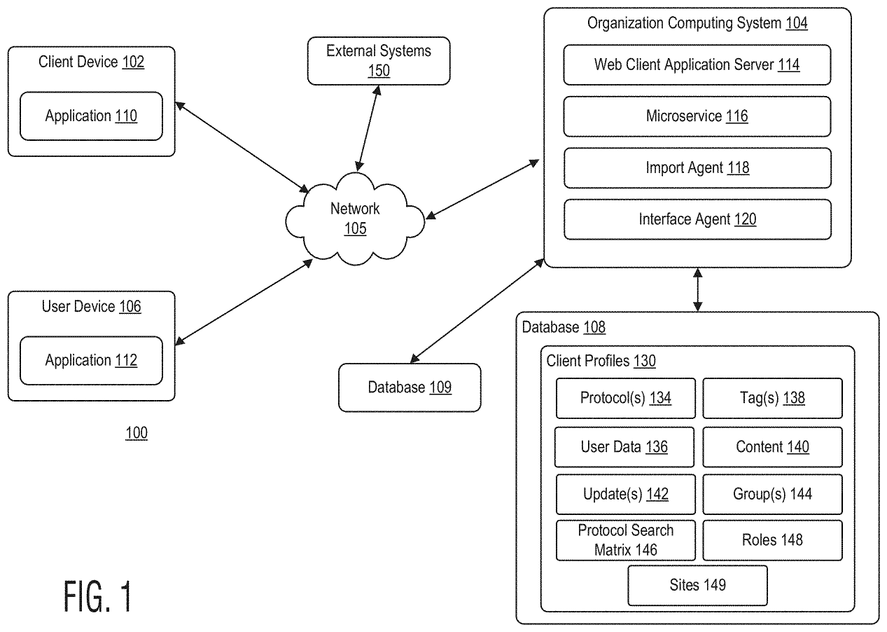

FIG. 1 is a block diagram illustrating a computing environment 100, according to example embodiments. Computing environment 100 may include client device 102, organization computing system 104, user device 106, and database 108 communicating via one or more networks 105.

Network 105 may be of any suitable type, including individual connections via the Internet, such as cellular or Wi-Fi networks. In some embodiments, network 105 may connect terminals, services, and mobile devices using direct connections, such as radio frequency identification (RFID), near-field communication (NFC), Bluetooth™, low-energy Bluetooth™ (BLE), Wi-Fi™ ZigBee™, ambient backscatter communication (ABC) protocols, USB, WAN, or LAN. Because the information transmitted may be personal or confidential, security concerns may dictate one or more of these types of connection be encrypted or otherwise secured. In some embodiments, however, the information being transmitted may be less personal, and therefore, the network connections may be selected for convenience over security.

Network 105 may include any type of computer networking arrangement used to exchange data or information. For example, network 105 may be the Internet, a private data network, virtual private network using a public network and/or other suitable connection(s) that enables components in computing environment 100 to send and receive information between the components of computing environment 100.

Client device 102 may be operated by a user. For example, client device 102 may be a mobile device, a tablet, a desktop computer, or any computing system having one or more of the capabilities described herein. Client device 102 may include at least one application 110. Application 110 may be representative of a web browser that allows access to a website or a stand-alone application. Client device 102 may access application 110 to access functionality of organization computing system 104. Client device 102 may communicate over network 105 to request a webpage or other information, for example, from web client application server 114 of organization computing system 104. For example, client device 102 may be configured to execute application 110 to access one or more functionalities of organization computing system 104. The content that is displayed to client device 102 may be transmitted from web client application server 114 to client device 102, and subsequently processed by application 110 for display through a display associated with client device 102.

In some embodiments, such as those in which user device 110 may be a mobile device, a user may work with application 110 offline. For example, a user may download or cache certain content such that the user can access the downloaded or cached content offline.

Organization computing system 104 may be representative of one or more computer systems associated with an organization. Organization computing system 104 may include web client application server 114, microservice 116, import agent 118, and interface agent 120. Each of microservice 116, import agent 118, interface agent 120, and handler 122 may be formed from one or more software modules. The one or more software modules may be collections of instructions stored on a media (e.g., memory associated with organization computing system 104) that represents a series of machine instructions (e.g., program code) that implements one or more algorithmic steps. Such machine instructions may be the actual computer code a processor associated with organization computing system 104 interprets to implement the instructions, or, alternatively, may be a higher-level coding of the instructions that are interpreted to obtain the actual computer code. The one or more software modules may also include one or more hardware components. One or more aspects of the algorithm may be performed by the hardware components (e.g., circuitry) itself, rather as a result of an instruction.

In some embodiments, organization computing system 104 may undergo a validation process to ensure that it achieves the highest security and privacy standards. For example, organization computing system 104 may be Good Clinical Practice validated. Good Clinical Practice validated is an international ethical, scientific, and practical standard to which all clinical research is conducted.

Import agent 118 may be configured to import various documents and information for a given protocol. For example, import agent 118 may be representative of a portable document format (PDF) protocol importer. Import agent 118 may be configured to convert various documents and information for a given protocol into a PDF format compatible with organization computing system 104. In some embodiments, import agent 118 may be configured to convert various documents and information into a searchable PDF format. In some embodiments, import agent 118 may handle documents and information that are not in a PDF format. For example, import agent 118 may be configured to handle and process the upload of video information or audible information associated with a protocol.

In some embodiments, import agent 118 may be configured to generate a hypertext markup language (HTML) text version of the protocol from the associated PDF. The HTML text version may be considered a hidden mirrored version of the protocol that sits behind the PDF. Such HTML text version may enable search functionality within the protocol, such as allowing search terms to be attached to content and targeted protocols automatically. In some embodiments, the HTML text version may be seen functioning when the user initiates a search. For example, as discussed in more detail below, the relevant word matches within the protocol may be highlighted, using the HTML text version.

In some embodiments, import agent 118 may be configured to dynamically tag the various documents and/or information uploaded by client device 102. For example, import agent 118 may embed one or more tags into various sections of the uploaded documents. By embedding one or more tags into various sections of the uploaded document, import agent 118 may allow for quick search both at the document level and, more broadly, at the protocol level. In some embodiments, import agent 118 may utilize one or more machine learning algorithms to understand, process, and tag various sections of the uploaded documents. In some embodiments, import agent 118 may allow a user to tag various sections of the uploaded documents. Upon receiving a tag for a given section of an uploaded document, import agent 118 may generate a record of the tag and store the record in database 108.

In some embodiments, import agent 118 may be configured to time stamp various portions of content. For example, a PDF document may be time stamped (e.g., automatically or manually) to the video, so that organization computing system 104 knows when to display additional material in time with the correct section of the video (e.g., automatic scrolling).

Interface agent 120 may be configured to generate one of more graphical user interfaces (GUIs) to be transmitted to client device 102 and/or user device 106 for rendering and presentation thereon. For example, interface agent 120 may format one or more GUIs associated with a portal for a given protocol. Interface agent 120 may generate one or more GUIs based on predefined templates. In some embodiments, the predefined templates may be selected by an administrator associated with organization computing system 104. In some embodiments, the predefined templates may be selected by an administrator associated with client device 102.

Microservice 116 may be configured to handle search queries from client device 102 or user device 106. Via an actionable element on a GUI generated by interface agent 120, a user associated with client device 102 or user device 106, respectively, may request microservice 116 to execute a query against the information associated with a protocol stored in database 108. Microservice 116 may execute the query against the protocol, by searching database 108 to identify potential search results based on the query and the protocol. In other words, microservice 116 may execute the query to determine which documents associated with the protocol are relevant to the query. In some embodiments, microservice 116 may be configured to execute the query within a document (e.g., using natural language processing to identify terms within the document that match the query). In some embodiments, microservice 116 may be configured to execute the query across documents associated with a protocol. For example, microservice 116 may utilize natural language processing, as well as the dynamically generated tags and/or user generated tags, to search across documents within a given protocol. In such embodiments, microservice 116 may be configured to return sections of a document or video clips or audio clips that satisfy the user's search criteria.

In some embodiments, search results may be categorized into three types, with all results being hyperlinked to the HTML mirrored version of the protocol. The three types of search results may include matched multimedia assets (e.g., “related content”), matched protocol sections or matched words (e.g., “related sections”), and word-match within the protocol (e.g., “found in protocol”). Matched multimedia assets may refer to results that are linked to search terms via a search matrix. For example, the search term “inclusion” may result in the video titled “Inclusion criteria.” Matched protocol sections or matched words may refer to results that are linked to search terms via the search matrix. For example, the search term “inclusion” may result in three sub-sections being linked, including the study design and inclusion criteria. In addition, results from linked related words to “inclusion” may be shown.

In some embodiments, microservice 116 may support predictive search functionality. For example, organization computing system 104 may include a search term dictionary. The search term dictionary helps microservice 116 match search terms to the protocol automatically. In some embodiments, user-based search terms may be collected within database 108 and stored as a protocol search matrix 146 (described below). In some embodiments, user search terms may be prioritized as a predictive result by user “role.” Microservice 116 may learn to improve the search results based on the search history for that user. For example, microservice 116 may include one or more machine learning techniques to learn each user's search habits. For example a nurse (i.e., a role) inputs a free text search term. Microservice 116 may predict search terms input by other users having the same role (i.e., other nurses) and may share predicted results that are likely to be most relevant. As output, microservice 116 may generate a ranked list of search terms by role and location.

In some embodiments, microservice 116 may include a search optimization process. For example, microservice 116 may tally all positive search queries per role in database 108. Such tallying may impact positively on the search experience for other users having the same role. For example, the trending positive search results may prioritize the predictive search results for those in the same role. In addition, all search queries without results (i.e., non-matching) may be tallied in database 108. Microservice 116 may not utilize the non-matching results. In some embodiments, administrators may manually review the non-matching search results and may assign a new key word to a section of a protocol or tag it to a piece of content. Once new keywords are added, further search utilizing these search terms may result in a positive search.

In some embodiments, organization computing system 104 may be configured to communicate with database 108. Database 108 may include one or more client profiles 130. Each client profile 130 may be associated with an entity corresponding to client device 102. Client profiles 130 may store various information associated with protocols generated by a given entity. For example, as illustrated, client profiles 130 may include, at least, one or more protocols 134 (hereinafter “protocol 134”), user data 136, one or more tags 138, content 140, updates 142, and groups 144. Each protocol 134 may correspond to a protocol for a clinical trial as defined by client profile 130. As provided above, each protocol 134 may define various content that define the objects or design of a clinical trial.

Content 140 may correspond to the various content that defines the objects or designs of a clinical trial. Content 140 may be representative of documents, audio files, video files, and the like, that contain information associated with the clinical trial. In some embodiments, prior to being stored in database 108, content 140 may be pre-processed by import agent 118.

In some embodiments, content 140 may include content in various languages and versions.

Tags 138 may correspond to one or more system-defined and/or user-defined tags associated with protocol 134. Each tag may correspond to a given section or portion of content 140. For example, a first tag may correspond to a first section of a document and a second tag may correspond to a first video clip. In some embodiments, both the first tag and the second tag may satisfy the same search query. In other words, the first tag and the second tag may be used to “mark up” content 140, such that content 140 is in a format that is more easily searchable.

User data 136 may correspond to user or member data for those users or members associated with the entity. User data 136 may include, for example, registration information corresponding to each user device 106. By maintaining registration information corresponding to each user device 106, organization computing system 104 may be able to track user progress during training for each protocol 134. For example, each user associated with a user device 106 may be provided with a unique uniform resource locator (URL), by which they may set up their user name and password. Once set up, an entity associated with client device 102 may monitor enrollment in protocol 134 and progress through protocol 134.

In some embodiments, user data 136 may include an indication of whether the user is a “web user” or a “single sign-on” (SSO) user. A web user may refer to those users who create user accounts with organization computing system 104 to log into and out of the platform. Administrators, e.g., user devices 106, may change user details and can assign web users to various groups. An SSO user may refer to those users who are authenticated via SSO functionality. In some embodiments, SSO users may have their user profile created automatically on first access. Administrators, e.g., user devices 106, may change user details and can assign SSO users to various groups.

In some embodiments, user data 136 may include a role 148 for each user (e.g., user device 106). Role 148 may be defined by an administrator, such as client device 102. In some embodiments, role 148 may be created or defined before the initiation of the study. By assigning the user a role 148, microservice 116 may segment users and define their protocol experience, including the management of various versions of the protocol.

In some embodiments, client profile 130 may include sites 149. Sites 149 may refer to investigator sites. Similar to roles 148, microservice 116 may use sites 149 to segment and manage segmentation and the overall user experience.

In some embodiments, each user profile 130 may include groups 144. Each group 144 may correspond to one or more users defined by user data 136. In some embodiments, each group 144 may be generated based on, in part, roles 148 and sites 149. In some embodiments, each group 144 may include a group identifier (e.g., “group ID”) associated therewith. As such, each user that includes the same group ID in user data 136 may correspond to a respective group as defined by client device 102. In this manner, organization computing system 104 can manage specific content and notifications to be delivered to certain groups of users. For example, clients can manage which set of sites or pages are shown to specific groups of users. In another example, clients can manage which geographic region of users can have access to certain resources. Exemplary notifications may include push notifications, emails, text messages, and the like.

Updates 142 may correspond to various updates that may be associated with protocol 134. For example, a user of client device 102 may provide various updates 142 to organization computing system 104. As such, instead of taking down content 140 that is already in use, through updates 142, organization computing system 104 may supplement content 140 that is already provided to end users. In this manner, client device 102 can seamlessly update protocol 134, without limiting access to end users.

In some embodiments, database 108 may include a protocol search matrix 146. Protocol search matrix 146 may be representative of a record of search requests for a given protocol. For example, protocol search matrix 146 may be representative of a spreadsheet that lists search terms and the corresponding protocol sections that result in these search terms. In some embodiments, protocol search matrix 146 may highlight or emphasize those search terms that were not expected, and identify how a user reacted when presented with the closest search results. Protocol search matrix 146 may be uploaded or integrated with microservice 116 for further refinement or development of the search functionality of microservice 116.

In some embodiments, microservice 116 may further be configured to support integration with various external systems 150. In some embodiments, microservice 116 may provide integration to various Electronic Data Capture (EDC) platforms (e.g., Medidata RAVE EDC. In some embodiments, microservice 116 may be configured to facilitate single sign-on (SSO) integration, such that credentials for accessing a protocol portal may be used to access various external system 150.

In some embodiments, organization computing system 104 may be configured to communicate with database 109. Database 109 may be configured to satisfy requirements or compliance with 21 CFR part 11 of the FDA Guidance for Industry. For example, database 109 may maintain a record of user engagements on the individual level and the group level.

User device 106 may be operated by a user having a relationship with an entity associated with client device 102. For example, user device 106 may be a mobile device, a tablet, a desktop computer, or any computing system having one or more of the capabilities described herein. User device 106 may include at least one application 112. Application 112 may be representative of a web browser that allows access to a website or a stand-alone application. User device 106 may access application 112 to access functionality of organization computing system 104. User device 106 may communicate over network 105 to request a webpage or other information, for example, from web client application server 114 of organization computing system 104. For example, user device 106 may be configured to execute application 112 to access one or more functionalities of organization computing system 104. The content that is displayed to user device 106 may be transmitted from web client application server 114 to user device 106, and subsequently processed by application 112 for display through a display associated with user device 106.

In some embodiments, such as those in which user device 106 may be a mobile device, a user may work with application 112 offline. For example, a user may download or cache certain content such that the user can access the downloaded or cached content offline.

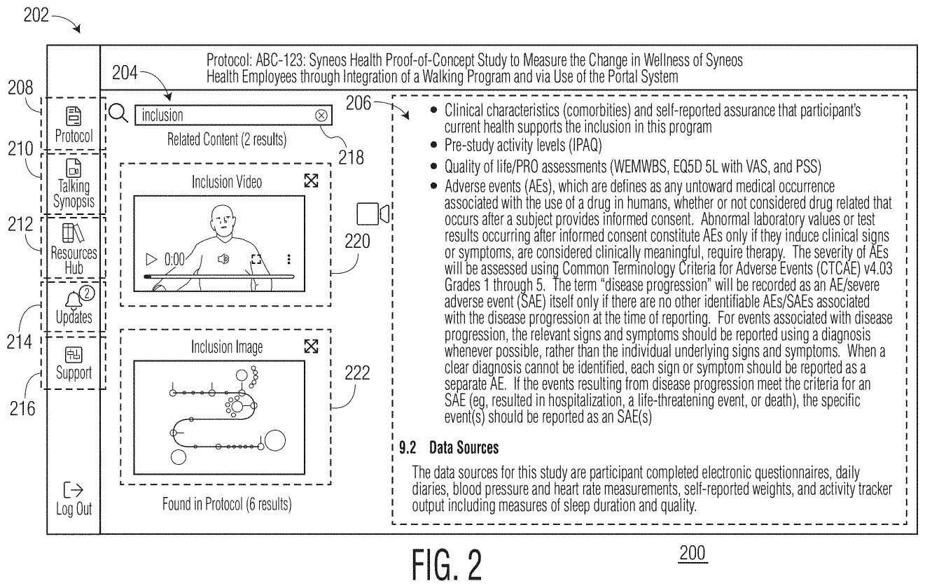

FIG. 2 illustrates an example view of a graphical user interface 200 presenting content 140 associated with a protocol 134, according to example embodiments. Graphical user interface 200 (hereinafter “GUI 200”) may correspond to a GUI generated by interface agent 120. In some embodiments, GUI 200 may be a web page presented in a web browser application (e.g., application 110) of client device 102. In some embodiments, GUI 200 may be a graphical user interface generated by a native software application (e.g., application 110) executing on client device 102.

As illustrated, GUI 200 may correspond to a webpage associated with a given protocol 134. GUI 200 may include at least a first portion 202, a second portion 204, and a third portion 206. First portion 202 may correspond to a navigation pane of GUI 200. First portion 202 may include one or more graphical elements 208-216 (e.g., buttons, links, etc.) selectable by the user to cause GUI 200 (e.g., the browser or native application) to send a message to organization computing system 104 requesting information associated with each graphical element 208-216.

Graphical element 208 may correspond to a protocol 134. Accordingly, upon receiving input via graphical element 208, GUI 200 may request, from organization computing system 104, all content 140 associated with protocol 134. Organization computing system 104 may deliver the content to GUI 200 so that GUI 200 may display the content on the display associated with user device 102.

Graphical element 210 may correspond to a talking synopsis. A talking synopsis may refer to a page, in which the end user may be provided with a video and/or audio recording summarizing content for protocol 134. In some embodiments, talking synopsis may further include written content corresponding to the synopsis. In some embodiments, written content may be configured to automatically scroll to correspond to current sections of the synopsis. In some embodiments, written content may include PDF versions of slides. In this matter, the end user may be provided with an overview of a relevant section of content and the section of content simultaneously. Accordingly, upon receiving input via graphical element 210, GUI 200 may request, from organization computing system 104, a talking synopsis corresponding to protocol 134.

Graphical element 212 may correspond to resources hub. Resources hub may include various content associated with a given protocol. Exemplary resources may include multiple formats, such as, but not limited to, video presentations from study experts, animated videos such as mode of action (MOA) explanations, presentations shared as a series of join photo graphic experts group (JPEG) images or portable document format (PDF), audio files, image files, PDF, and content hosted online external to organization computing system 104. Accordingly, upon receiving input via graphical element 212, GUI 200 may request, from organization computing system 104, all resources corresponding to protocol 134.

Graphical element 214 may correspond to updates for a protocol. For example, as recited above, a client device 102 may upload various updates to a protocol 134. Upon receiving and processing an update, organization computing system 104 may update graphical element 214, such that graphical element 214 visually denotes that an update is available. As illustrated, graphical element 214 includes a number “2” signaling to the end user that two updates are available for protocol 134. Upon receiving input via graphical element 214, GUI 200 may request, from organization computing system 104, all available updates to protocol 134. In some embodiments, graphical element 214 may include an indicator, such as a counter, that provides a visual indication regarding when a new update is added. For example, when a new update is available, graphical element 214 may include a “1” superimposed thereon. In another example, graphical element 214 may include another visual indicator (e.g., a bell) that may visually indicate a new update.

Graphical element 216 may correspond to support. Support may be customized to the needs of the study. For example, an instructional video may be made available, which could include study specific instructions. In some embodiments, contact information for those that are relevant to the user will be provided.

Second portion 204 may correspond to a search section of GUI 200. For example, second portion 204 may be displayed upon an end user interacting with graphical element 208. Second portion 204 may include graphical element 218. Graphical element 218 may correspond to a search bar. Via graphical element 218, an end user can submit search requests to organization computing system 104. In some embodiments, second portion 204 may further include one or more graphical elements 220 and 222. Graphical elements 220 and 222 may correspond to search results based on the received query.

In operation, upon receiving a search request via graphical element 218, GUI 200 may submit the search request to organization computing system 104. Microservice 116 of organization computing system 104 may subsequently search database 108 based on the search request. For example, microservice 116 may query database 108 to determine search results that match the search request. In some embodiments, querying database 108 may involve identifying one or more tags 138 that satisfy the search request. By identifying one or more tags 138, microservice 116 searches across content associated with protocol 134. In some embodiments, microservice 116 may search within content 140. For example, microservice 116 may search within a displayed item of content using one or more natural language processes. In this manner, microservice 116 may search within content and across content based on the search request.

As shown, graphical element 220 may correspond to video content; graphical element 222 may correspond to image content. Each of graphical element 220 and graphical element 222 may be expandable by an end user.

Third portion 206 may correspond to a display section of GUI 200. For example, upon the user interacting with graphical element 208, graphical element 210, graphical element 212, graphical element 214, or graphical element 216, GUI 200 may display content associated with the selection in third portion 206. As illustrated, user of user device 106 may have interacted with graphical element 208 corresponding to a protocol. Accordingly, third portion 206 may include content associated with the protocol.

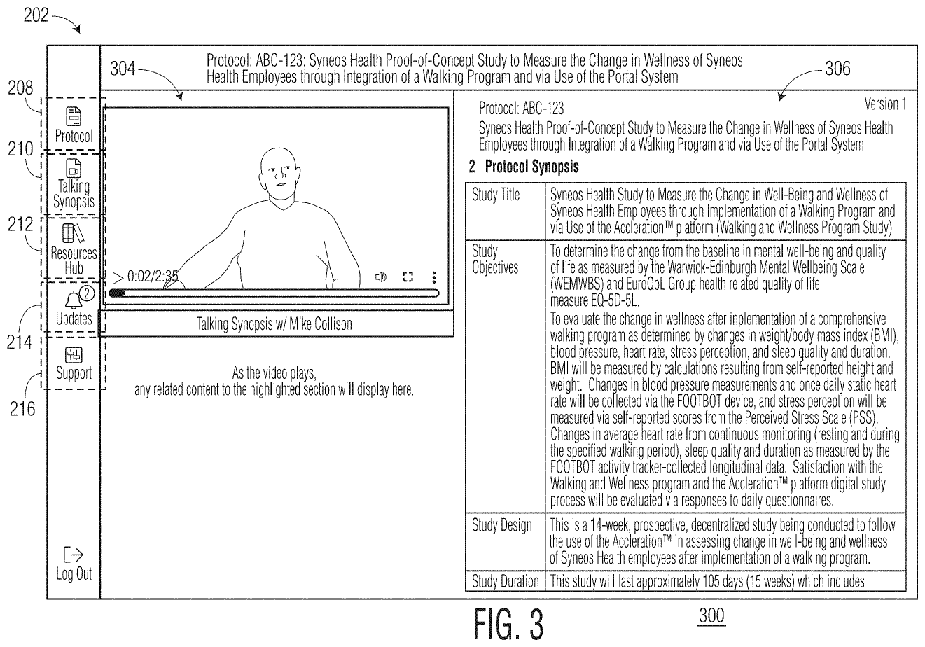

FIG. 3 illustrates an example view of a graphical user interface 300 presenting a talking synopsis of a protocol 134, according to example embodiments. Graphical user interface 300 (hereinafter “GUI 300”) may correspond to a GUI generated by interface agent 120 in response to an end user selecting graphical element 210 via GUI 200. In some embodiments, GUI 300 may be a web page presented in a web browser application (e.g., application 110) of client device 102. In some embodiments, GUI 200 may be a graphical user interface generated by a native software application (e.g., application 110) executing on client device 102.

As illustrated, GUI 300 may include second portion 304 and third portion 306. Second portion 304 may include video content. For example, second portion 304 may include a video that provides a synopsis of content associated with protocol 134. As illustrated, second portion 304 notifies end users that “As the video plays, related content to the highlighted section will display here.”

Third portion 306 may include content 140 associated with protocol 134. Third portion 306 may include dynamic content that updates, depending on a current portion of the video. In some embodiments, content provided in third portion 306 may scroll automatically to the relevant section. In this manner, the user can be provided with a portion of content that is currently being explained in the video provided in second portion 304.

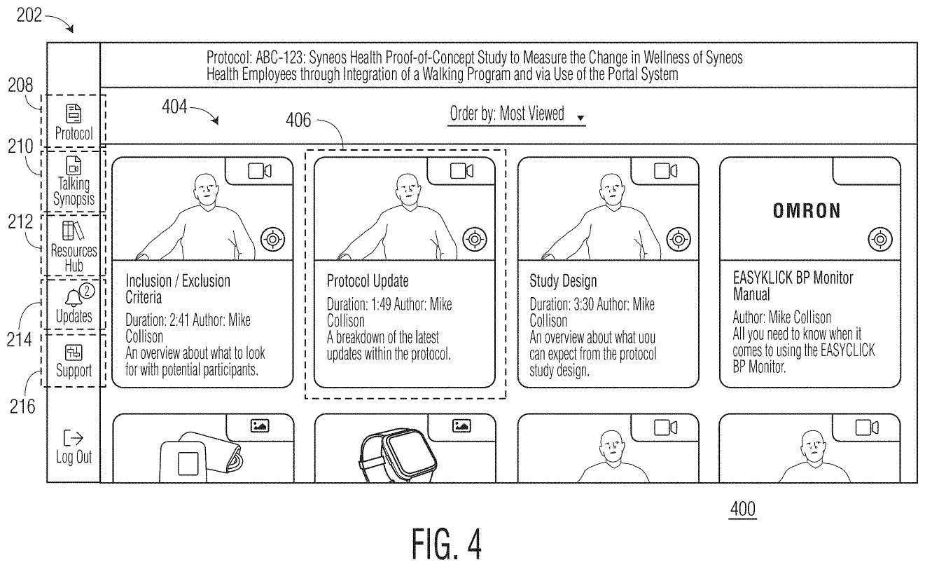

FIG. 4 illustrates an example view of a graphical user interface 400 presenting a resources page for a protocol 134, according to example embodiments. Graphical user interface 400 (hereinafter “GUI 400”) may correspond to a GUI generated by interface agent 120 in response to an end user selecting graphical element 212 via GUI 200. In some embodiments, GUI 400 may be a web page presented in a web browser application (e.g., application 112) of user device 106. In some embodiments, GUI 400 may be a graphical user interface generated by a native software application (e.g., application 112) executing on user device 106.

As illustrated, GUI 400 may include second portion 404. Second portion 404 may include one or more graphical elements 406. Each graphical element 406 may correspond to an item of content in resource hub. For example, as illustrated, each graphical element 406 may correspond to one or more of a document (e.g., PDF document), a video, an audio clip, and image, etc. Interface agent 120 may be configured to generate GUI 400 using content 140 and tags 138 associated with a given protocol 134.

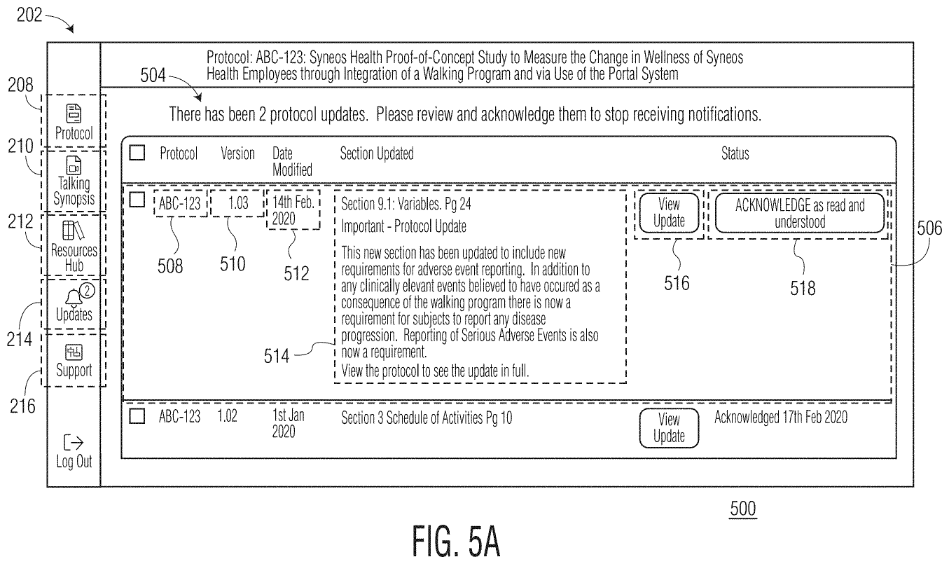

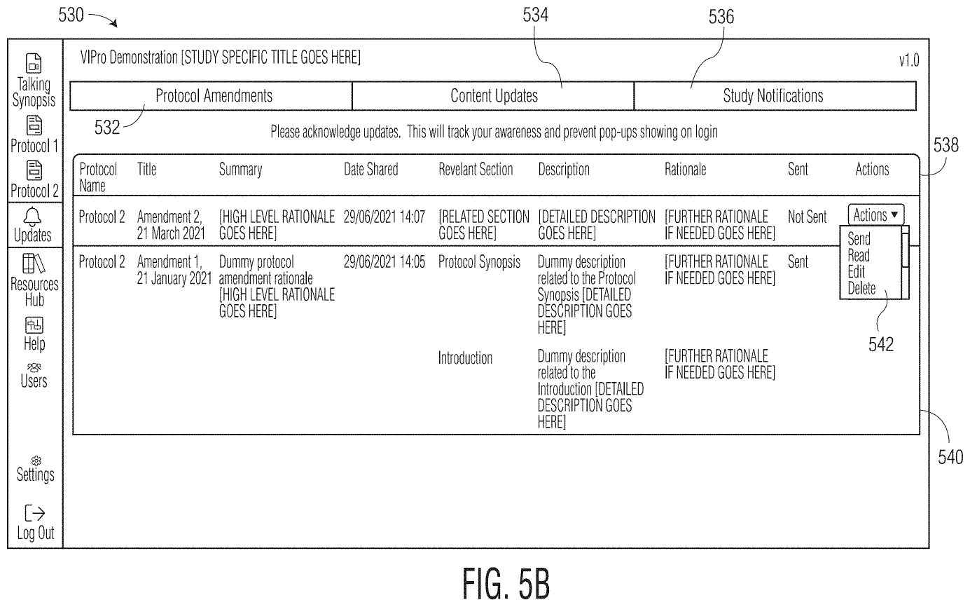

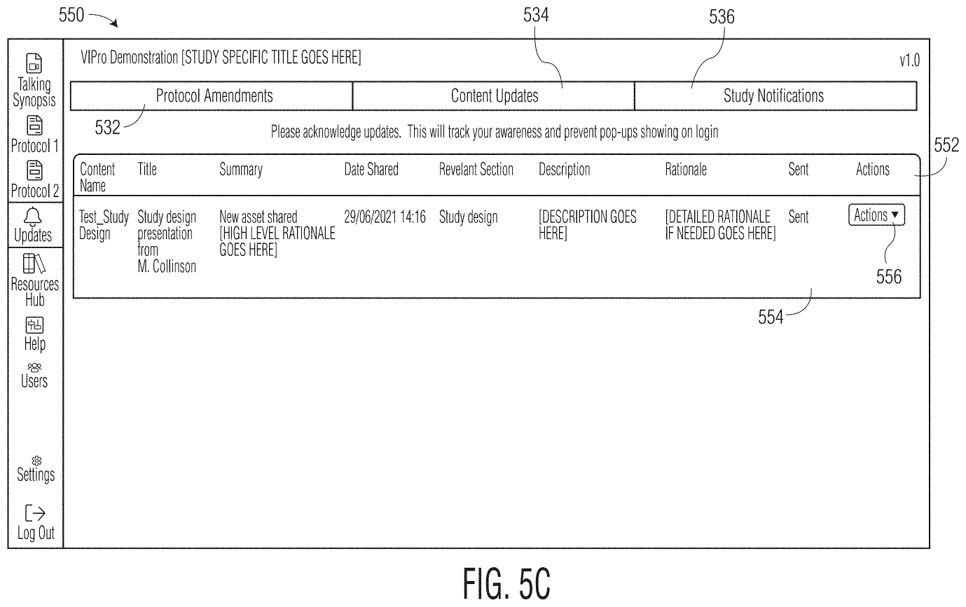

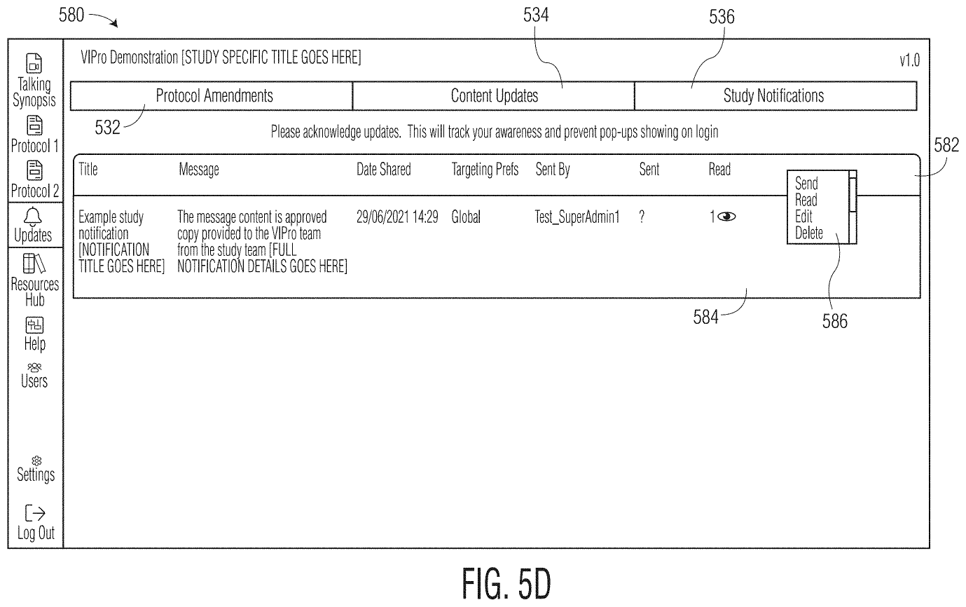

FIG. 5A illustrates an example view of graphical user interface 500 presenting updates associated with a protocol 134, according to example embodiments. Graphical user interface 500 (hereinafter “GUI 500”) may correspond to a GUI generated by interface agent 120 in response to an end user selecting graphical element 214 via GUI 200. In some embodiments, GUI 500 may be a web page presented in a web browser application (e.g., application 112) of user device 106. In some embodiments, GUI 500 may be a graphical user interface generated by a native application (e.g., application 112) executing on user device 106.

As illustrated, GUI 500 may include second portion 504. Second portion 504 may include one or more graphical elements 506. Each graphical element 506 may correspond to a given update. Graphical element 506 may include protocol ID 508, version number 510, modification date 512, update description 514, actionable element 516, and actionable element 518. Protocol ID 508 may correspond to a unique ID associated with the selected protocol. Version number 510 may correspond to the updated version. Modification date 512 may correspond to the date of the update. Update description 514 may describe changes associated with the update. Such description may include an indication of the relevant sections that are affected by the update. Actionable element 516 may correspond to a URL, which, when activated, may direct the end user to relevant sections of content associated with the update. Actionable element 518 may correspond to an interactive element that, when activated, signifies user acknowledgement of the update.

In some embodiments, if the user is viewing GUI 500 for the first time following an update, GUI 500 may include an overlay element that notifies the user or administrator of new content.

FIG. 5B illustrates an example view of a graphical user interface 530 presenting updates associated with a protocol 134, according to example embodiments. Graphical user interface 530 (hereinafter “GUI 530”) may correspond to a GUI generated by interface agent 120 in response to an end user selecting graphical element 214 via GUI 200. In some embodiments, GUI 530 may be a web page presented in a web browser application (e.g., application 112) of user device 106. In some embodiments, GUI 530 may be a graphical user interface generated by a native application (e.g., application 112) executing on user device 106.

As illustrated, GUI 530 may include various headers such as protocol amendments header 532, content updates header 534, and study notifications header 536. Protocol amendments header 532 may correspond to a listing of amendments or changes to a given protocol, such as protocol 134. Content updates header 534 may provide a listing of updates to content associated with a given protocol. Study notifications header 536 may correspond to notification regarding a study associated with a given protocol. GUI 530 may correspond to an interface generated in response to a user selecting protocol amendments header 532.

As shown, GUI 530 may include a listing 538 of protocol amendments 540. Each protocol amendment 540 may include various information corresponding thereto. For example, each protocol amendment 540 may include one or more of: a protocol name, a title, summary, date shared, relevant section, description, rationale, sent indication, or actions indication. Protocol name may be associated with a name for the protocol to which a respective protocol amendment 540 corresponds. Title may be associated with a title for the protocol to which a respective protocol amendment 540 corresponds. Summary may provide a brief overview of a respective protocol amendment 540. Date shared may be representative of a date associated with the generation or sharing of a respective protocol amendment. Relevant section may indicate which section of the protocol a respective protocol amendment 540 corresponds. Rationale may correspond to the reason the protocol amendment 540 was generated. Sent indication may correspond to whether a notification of the protocol amendment 540 was sent. Actions may include a graphical item that allows the user to take a respective action. For example, as shown, actions may include a dropdown menu 542 that allows the user to send a notification, read the protocol amendment 540, edit the protocol amendment, or delete the protocol amendment.

FIG. 5C illustrates an example view of a graphical user interface 550 presenting updates associated with a protocol 134, according to example embodiments. Graphical user interface 550 (hereinafter “GUI 550”) may correspond to a GUI generated by interface agent 120 in response to an end user selecting content updates header 534. In some embodiments, GUI 550 may be a web page presented in a web browser application (e.g., application 112) of user device 106. In some embodiments, GUI 550 may be a graphical user interface generated by a native application (e.g., application 112) executing on user device 106.

As illustrated, GUI 550 may include a listing 552 of content updates 554. Each content update 554 may include various information corresponding thereto. For example, each content update 554 may include one or more of: a content name, a title, summary, date shared, relevant section, description, rationale, sent indication, or actions indication. Content name may be associated with a name for the new content to which a respective content update 554 corresponds. Title may be associated with a title for the new content to which a respective content update 554 corresponds. Summary may provide a brief overview of a respective content update 554. Date shared may be representative of a date associated with the generation or sharing of a respective content update 554. Relevant section may indicate which section of the protocol a respective content update 554 corresponds. Rationale may correspond to the reason a respective content update 554 was generated. Sent indication may correspond to whether a notification for a respective content update 554 was sent. Exemplary notifications may include push notifications, emails, text messages, and the like. Actions may include a graphical item that allows the user to take a respective action. For example, as shown, actions may include a dropdown menu 556 that allows the user to send a notification, read a respective content update 554, edit a respective content update 554, or delete a respective content update 554.

FIG. 5D illustrates an example view of a graphical user interface 580 presenting study notifications associated with a protocol 134, according to example embodiments. Graphical user interface 580 (hereinafter “GUI 580”) may correspond to a GUI generated by interface agent 120 in response to an end user selecting study notifications header 536. In some embodiments, GUI 580 may be a web page presented in a web browser application (e.g., application 112) of user device 106. In some embodiments, GUI 580 may be a graphical user interface generated by a native application (e.g., application 112) executing on user device 106.

As illustrated, GUI 580 may include a listing 582 of study notifications 584. Each study notification 584 may include various information corresponding thereto. For example, each study notification 584 may include one or more of: a title, a message, date shared, targeting preferences, sent by indication, sent count, read count, or actions indication. Title may correspond to a title of a respective study notification 584. Message may correspond to a message from a study team that may be provided with study notification 584. Date shared may be representative of a date associated with the generation or sharing of a respective study notification 584. Targeting preferences may correspond to a target demographic or group for a respective study notification 584. Sent by indication may correspond to a user or administrator that sent a respective study notification 584. Sent count may indicate a count of users to which a respective study notification 584 was sent. Read count may indicate a count of users that read a respective study notification 584. Actions may include a graphical item that allows a user or administrator to take a respective action. For example, as shown, actions may include a dropdown menu 586 that allows a user or administrator to send a notification, read a respective study notification 584, edit a respective study notification 584, or delete a respective study notification 584.

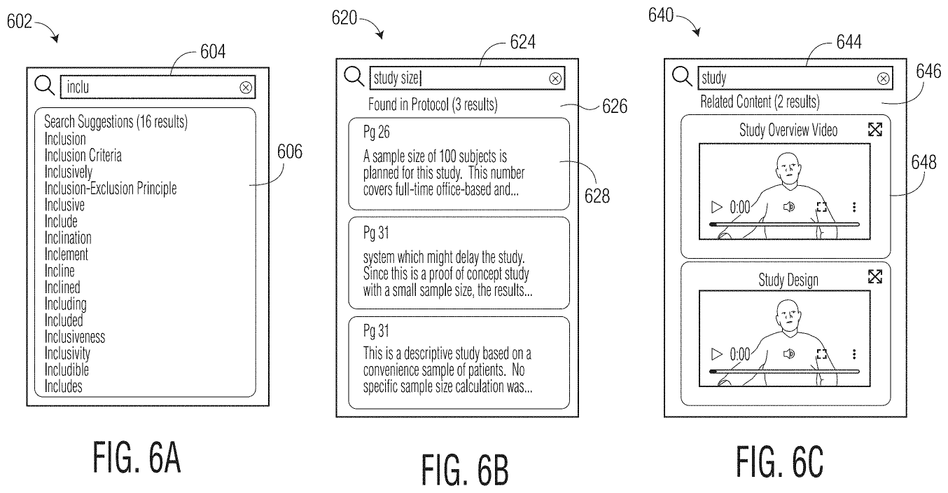

FIG. 6A illustrates an example view of a searching portion 602 of a graphical user interface, according to example embodiments. For example, searching portion 602 may correspond to second portion 204 of GUI 200.

As illustrated, second portion 602 may include a graphical element 604 and result section 606. Graphical element 604 may be representative of a search bar. For example, via graphical element 604, a user may submit a search query to organization computing system 104. In some embodiments, the search query may be directed to a local search within content displayed on a screen of user device 106. In some embodiments, the search query may be directed to a search across all content 140 associated with a protocol 134. For example, search query may be directed to video synopsis, resource hub, locally within displayed content, and/or updates.

As shown, as the user provides graphical element 604 with search terms, result section 606 may dynamically provide end users with suggested search terms. For example, as the user is typing “inclu,” the user may be provided with suggested search terms, such as, but not limited to, inclusion, inclusion criteria, inclined, and the like. In operation, upon receiving input via graphical element 604, application 112 may submit the query request to organization computing system 104. Microservice 116 may analyze characters in the search term and generate a plurality of search term options based on the characters provided by application 112.

FIG. 6B illustrates an example view of a searching portion 620 of a graphical user interface, according to example embodiments. For example, searching portion 620 may correspond to second portion 204 of GUI 200.

As illustrated, second portion 620 may include a graphical element 624 and result section 626. Graphical element 624 may be representative of a search bar. For example, via graphical element 624, a user may submit a search query to organization computing system 104. In some embodiments, the search query may be directed to a local search within content displayed on a screen of user device 106. In some embodiments, the search query may be directed to a search across all content 140 associated with a protocol 134. For example, search query may be directed to video synopsis, resource hub, locally within displayed content, and/or updates.

As shown, second portion 620 may receive a search request via graphical element 624. User device 106 may transmit the search request to organization computing system 104 via application 112 executing thereon. Upon receipt of the search request, microservice 116 may execute a query against database 108 to identify results for the search term. As illustrated, microservice 116 may execute a query that includes the phrase “study size” against database 108 to identify content corresponding to study size.

Result section 626 may include one or more graphical elements 628. Each graphical element 628 may correspond to a search result, based on the received search request. As illustrated, the search term “study size” may result in at least three search results.

FIG. 6C illustrates an example view of a searching portion 640 of a graphical user interface, according to example embodiments. For example, searching portion 640 may correspond to second portion 204 of GUI 200.

As illustrated, second portion 640 may include a graphical element 644 and result section 646. Graphical element 644 may be representative of a search bar. For example, via graphical element 644, a user may submit a search query to organization computing system 104. In some embodiments, the search query may be directed to a local search within content displayed on a screen of user device 106. In some embodiments, the search query may be directed to a search across all content 140 associated with a protocol 134. For example, search query may be directed to video synopsis, resource hub, locally within displayed content, and/or updates.

As shown, second portion 640 may receive a search request via graphical element 644. User device 106 may transmit the search request to organization computing system 104 via application 112 executing thereon. Upon receipt of the search request, microservice 116 may execute a query against database 108 to identify results for the search term. As illustrated, microservice 116 may execute a query that includes the term “study” against database 108 to identify content corresponding to study size.

Result section 646 may include one or more graphical elements 648. Each graphical element 648 may correspond to a search result, based on the received search request. As illustrated, each graphical element 648 may correspond to a video associated with the term “study.” In this manner, a user can be presented with video data in response to the query in addition to documents, images, and the like.

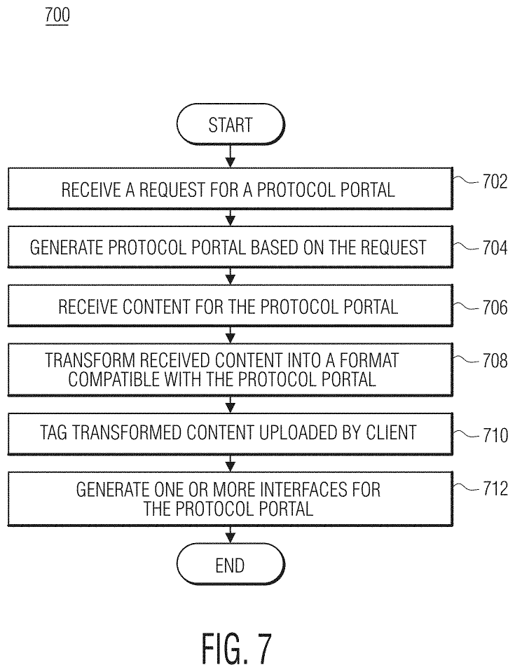

FIG. 7 is a flow diagram illustrating a method 700 of generating a protocol portal, according to example embodiments. Method 700 may begin at step 702.

At step 702, a client may request a protocol portal. For example, client device 102 may transmit a protocol portal request to organization computing system 104. In some embodiments, client device 102 may transmit the portal request to organization computing system 104 via application 110 executing thereon.

At step 704, organization computing system 104 may generate a protocol portal for the client. For example, organization computing system 104 may generate one or more protocol portal templates. In this manner, a client can easily upload various content associated with a protocol to organization computing system 104, without having to build the portal themselves.

At step 706, organization computing system 104 may receive content for a given protocol. In some embodiments, organization computing system 104 may receive content for the protocol via application 110 executing on client device 102.

At step 708, organization computing system 104 may transform the received content into a format compatible with the protocol portal. For example, import agent 118 may be configured to transform the received content into the format compatibly with protocol portal. Import agent 118 may store the transformed content in database 108. In some embodiments, import agent 118 may be representative of a portable document format (PDF) protocol importer. Import agent 118 may be configured to convert various documents and information for a given protocol into a PDF format compatible with organization computing system 104. In some embodiments, import agent 118 may be configured to convert various documents and information into a searchable PDF format. In some embodiments, import agent 118 may handle documents and information that are not in a PDF format. For example, import agent 118 may be configured to handle and process the upload of video information or audible information associated with a protocol.

At step 710, organization computing system 104 may tag content uploaded by client device 102. In some embodiments, import agent 118 may be configured to dynamically tag the various documents and/or information uploaded by client device 102. For example, import agent 118 may embed one or more tags into various sections of the uploaded documents. By embedding one or more tags into various sections of the uploaded document, import agent 118 may allow for quick search both at the document level and, more broadly, at the protocol level. In some embodiments, import agent 118 may utilize one or more machine learning algorithms to understand, process, and tag various sections of the uploaded documents. In some embodiments, import agent 118 may allow a user to tag various sections of the uploaded documents. Upon receiving a tag for a given section of an uploaded document, import agent 118 may generate a record of the tag and store the record in database 108.

At step 712, organization computing system 104 may generate one or more interfaces for the protocol portal. For example, interface agent 120 may be configured to generate one or more GUIs to be transmitted to client device 102 and/or user device 106 for rendering and presentation thereon. For example, interface agent 120 may format one or more GUIs associated with a portal for a given protocol. Interface agent 120 may generate one or more GUIs based on predefined templates. In some embodiments, the predefined templates may be selected by an administrator associated with organization computing 104. In some embodiments, the predefined templates may be selected by an administrator associated with client device 102.

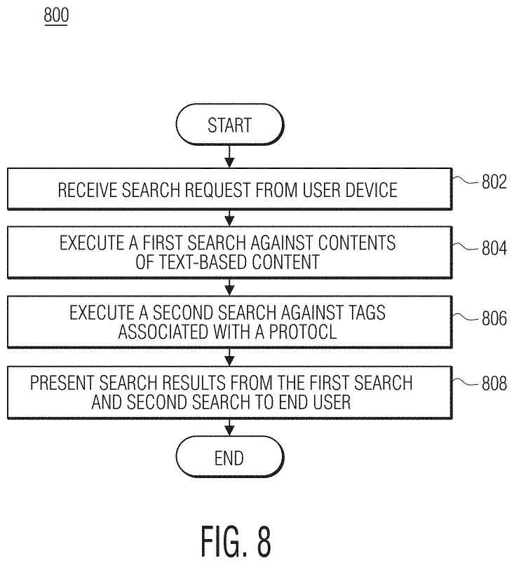

FIG. 8 is a flow diagram illustrating a method 800 of generating search results, according to example embodiments. Method 800 may begin at step 802.

At step 802, organization computing system 104 may receive a search request from user device 106. In some embodiments, organization computing system 104 may receive the search request from user device 106 via application 112 executing thereon. In some embodiments, search request may include a single term. In some embodiments, search request may include multiple terms, i.e., a search phrase.

At step 804, organization computing system 104 may execute a first search against contents of text-based content. For example, microservice 116 may execute a first search using one or more natural language processes to determine if any content in database 108 includes the term or terms provided by the user.

At step 806, organization computing system 104 may execute a second search against tags associated with a protocol. For example, microservice 116 may execute a second search using one or more natural language processes to determine if any tags in database 108 satisfies the term or terms provided by the user.

At step 808, organization computing system 104 may present the search results to the user. In some embodiments, search results may include text-based documents. In some embodiments, search results may include video content. In some embodiments, search results may include image content. In some embodiments, search results may include audio content.

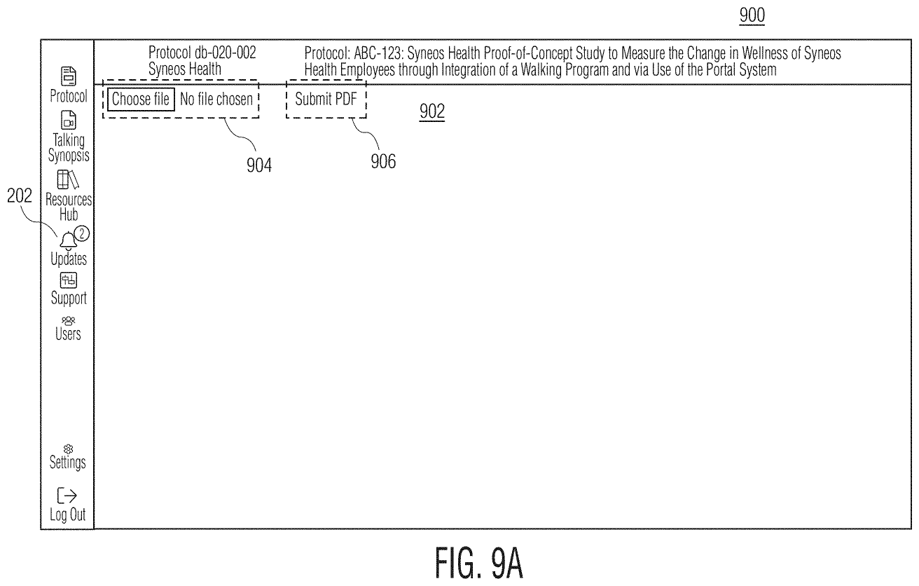

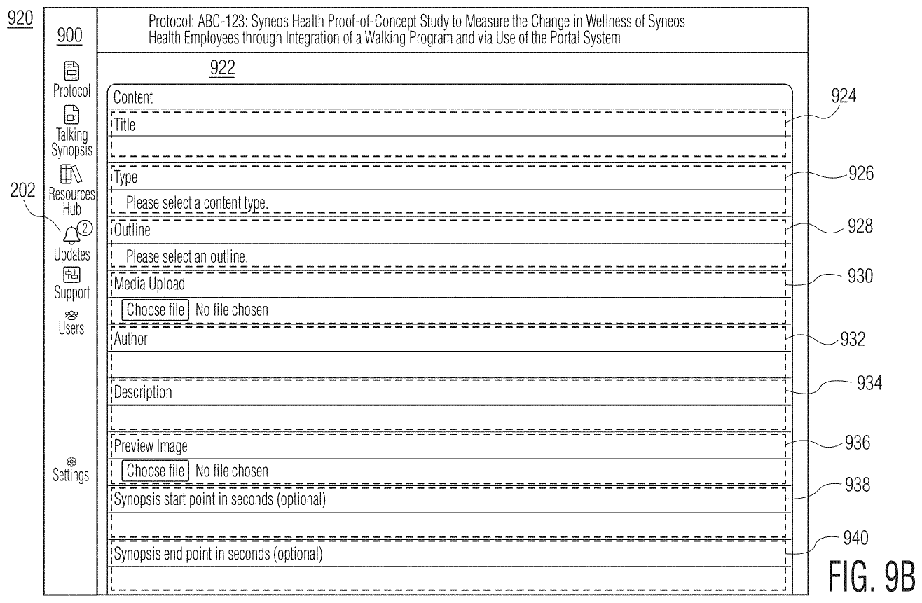

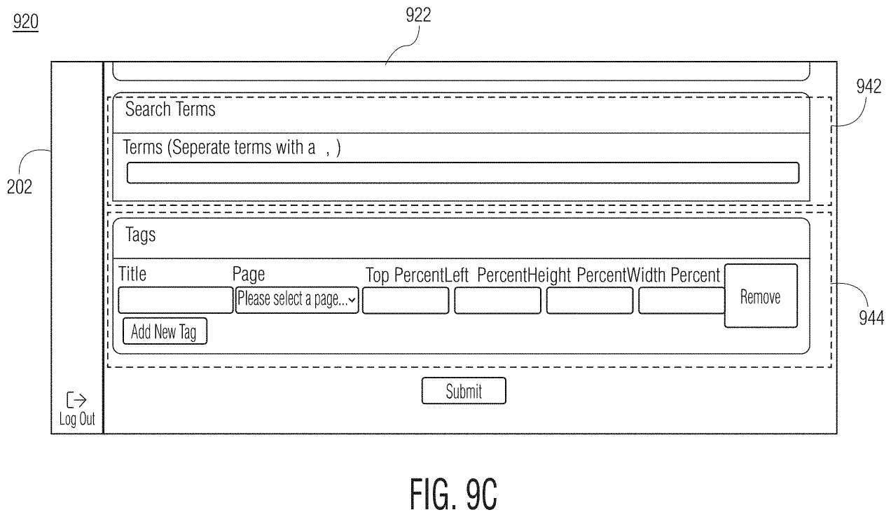

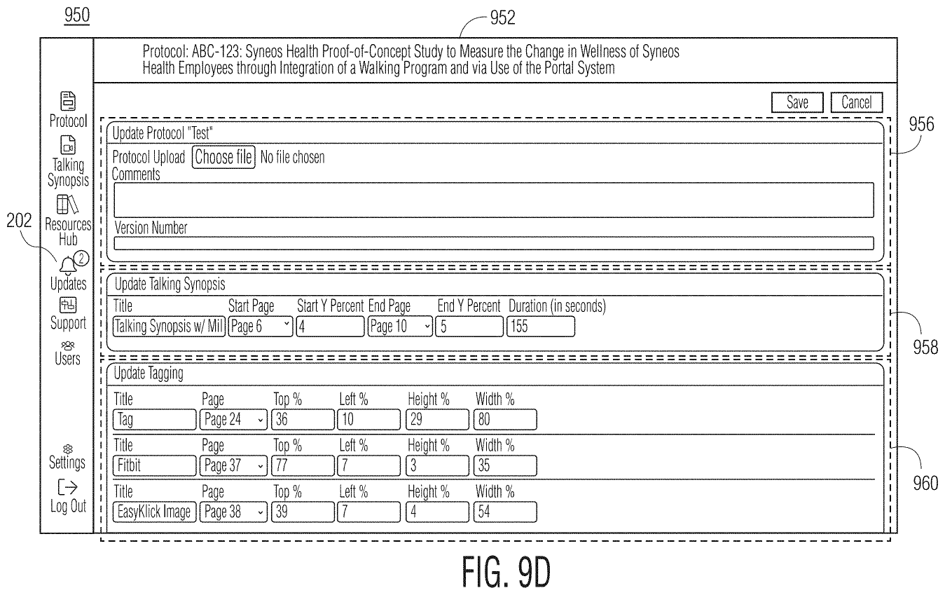

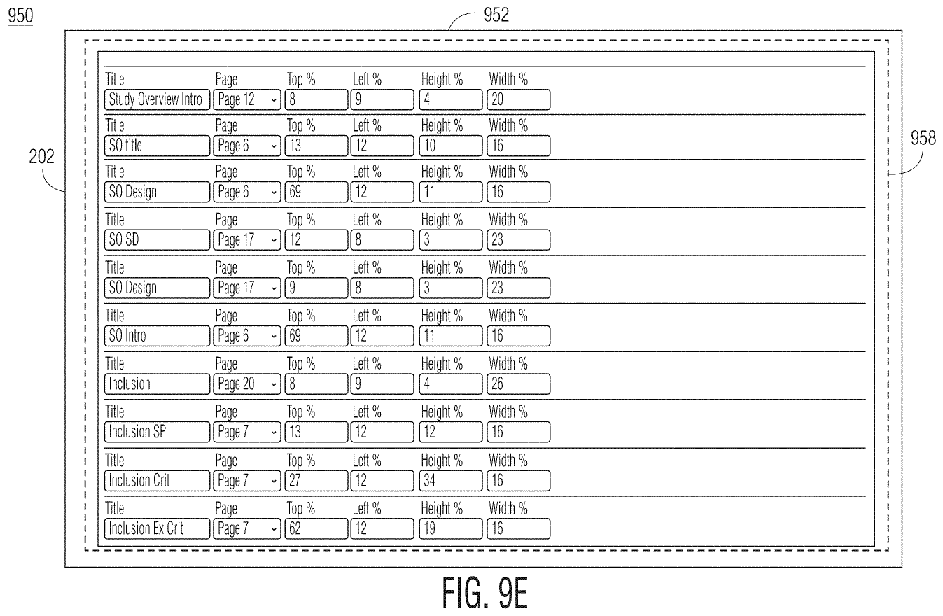

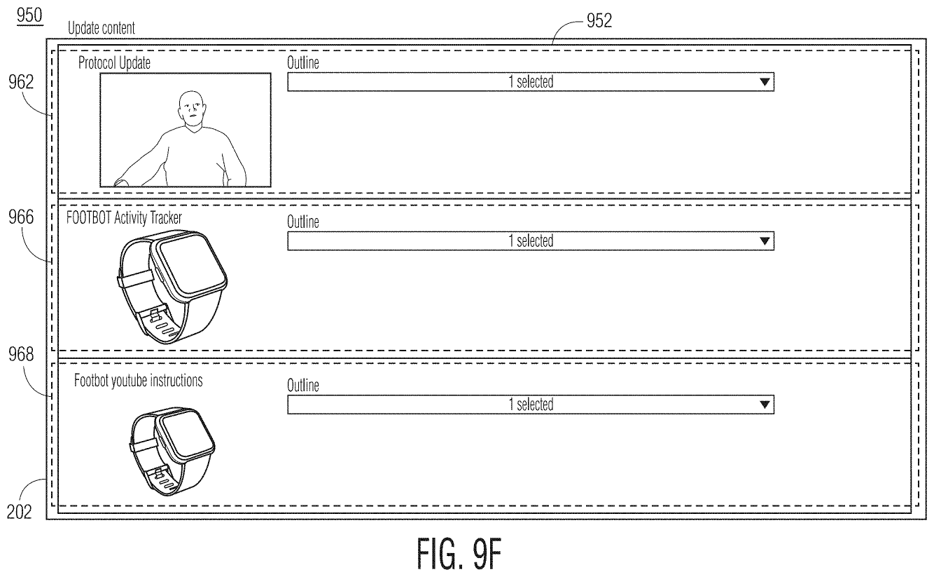

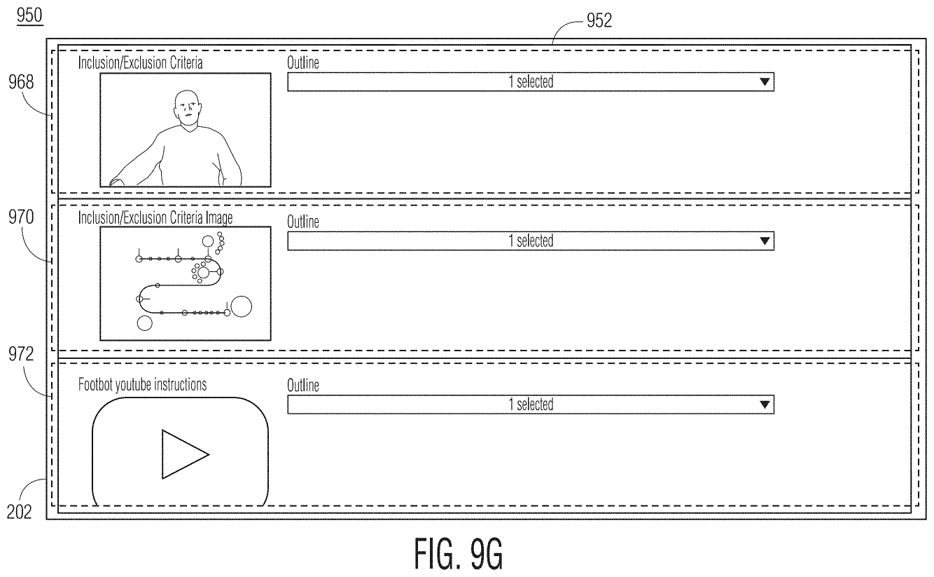

FIG. 9A illustrates an example view of a graphical user interface 900 through which a user can upload content 140 for a protocol 134, according to example embodiments. Graphical user interface 900 (hereinafter “GUI 900”) may correspond to a GUI generated by interface agent 120. In some embodiments, GUI 900 may be a web page presented in a web browser application (e.g., application 110) of client device 102. In some embodiments, GUI 900 may be a graphical user interface generated by a native software application (e.g., application 110) executing on client device 102.

As illustrated, GUI 900 may correspond to a content upload page associated with a given protocol 134. GUI 900 may include at least a first portion 202 and second portion 902. Second portion 902 may include graphical elements 904 and 906. Graphical element 904 may correspond to a “choose file” option. For example, when a user interacts with graphical element 904, client device 102 may present a user with a local file directory. Via local file directory, a user, via GUI 900, may select content for upload to organization computing system 104. Graphical element 906 may correspond to a “submit content” option. For example, after a user selects content for upload via a local file directory, the user may interact with graphical element 906 to upload the content to organization computing system 104 for inclusion in protocol 134.

FIG. 9B illustrates an example view of a graphical user interface 920 through which a client can upload content 140 for a protocol 134, according to example embodiments. Graphical user interface 920 (hereinafter “GUI 920”) may correspond to a GUI generated by interface agent 120. In some embodiments, GUI 920 may be a web page presented in a web browser application (e.g., application 110) of client device 102. In some embodiments, GUI 900 may be a graphical user interface generated by a native software application (e.g., application 110) executing on client device 102. In some embodiments, a user may be presented with GUI 920 following a submission of content via GUI 900.

As illustrated, GUI 920 may correspond to a content upload page associated with a given protocol 134. For example, via GUI 920, a user may provide additional information about a selected item of content for upload. GUI 920 may include at least a first portion 202 and second portion 922. Second portion 922 may include one or more fields 924-940. Field 924 may correspond to a title field. For example, via field 924, a user may define a title of the content to be uploaded. Field 926 may correspond to a type field. In some embodiments, field 926 may be represented as a dropdown menu. For example, via the dropdown menu of field 926, a user may select a content type that best corresponds to the content to be uploaded. Exemplary content types may include, but are not limited to, image, video, PDF, link, download link, etc. Field 928 may correspond to an outline field. In some embodiments, field 928 may be represented as a dropdown menu. In some embodiments, field 928 may provide a dropdown menu that allows an alignment of the content to a relevant section within protocol 134. Field 930 may correspond to a media upload field. For example, via field 930 a user may be able to upload various media, such as, but not limited to images, videos PDFs, downloaded, files, etc. In some embodiments, the media may be uploaded as a link (e.g., URL) to the media. In some embodiments, field 930 may include an actionable element, which, when selected by the user, displays a local file system of the client device 102. Field 932 may correspond to an author field. For example, via field 932, the user may add authorship information for the content to be uploaded. Field 934 may correspond to a description field. For example, via field 934, a user may provide a brief description of the content to be uploaded. Field 936 may correspond to a preview image field. For example, via field 936, a user may be able to upload a preview image corresponding to the content to be uploaded. In some embodiments, field 936 may include an actionable element, which, when selected by the user, displays a local file system of client device 102. Fields 938 and field 940 may correspond to a synopsis starting point and end point fields. For example, via fields 938 and 940, a user may be able to define various portions of content (e.g., starting and ending points) that are highly relevant. In some embodiments, such as those when the content to be uploaded is a video, the user may define a starting time (e.g., via field 938) and an ending time (e.g., via field 940) of highly relevant section(s).

FIG. 9C is a continuation of graphical user interface 920 through which a client can upload content 140 for a protocol 134, according to example embodiments. As further illustrated, second portion 902 may further include one or more fields 942 and 944. Field 942 may correspond to a terms field. For example, via field 942, a user may enter a list of words that, if searched, will trigger display of the content to be uploaded. Field 944 may correspond to a tags field. For example, via field 944, a user may define various tags that may be used to display relevant information within the content. In some embodiments, a tag may include: a title, a page number, and HTML coordinates (e.g., top percentage, left percentage, height percentage, and width percentage).

FIG. 9D illustrates an example view of a graphical user interface 950 through which a client can update a protocol 134, according to example embodiments. Graphical user interface 950 (hereinafter “GUI 950”) may correspond to a GUI generated by interface agent 120. In some embodiments, GUI 950 may be a web page presented in a web browser application (e.g., application 110) of client device 102. In some embodiments, GUI 950 may be a graphical user interface generated by a native software application (e.g., application 110) executing on client device 102.

As illustrated, GUI 950 may correspond to a content upload page associated with a given protocol 134. GUI 950 may include at least a first portion 202 and second portion 952. Second portion 952 may include one or more sections 956-960.

Generally, if any changes are made to protocol 134, new content may need to be uploaded. This may be done via Update Protocol page, which is illustrated in GUI 950. Section 956 may correspond to an “Update Protocol ‘Test’” section. Section 956 may, for example, allow a user to upload new content, add comments to the content, and include a version number of the update. In some embodiments, the comments added to the comments section may be displayed to end users via the update section of the protocol.

Section 958 may correspond to an “Update Talking Synopsis” section. For example, via section 958, a user may define the talking synopsis associated with the update. This may include, for example, a title, a start page, a start percent, an end page, an end percent, and a duration.

Section 960 may correspond to an “Update Tagging” section. Section 960 may allow the user to update HTML coordinates for the update.

In some embodiments, the user may be presented with an option to preview an added material for a compliance check before rolling out the updated information.

FIG. 9E is a continuation of graphical user interface 950 through which a client can upload content 140 for a protocol 134, according to example embodiments. As further illustrated, second portion 902 may include section 960 (continued from FIG. 9D above). Via section 960, a user may define HTML coordinates for the update.

FIG. 9F is a continuation of graphical user interface 950 through which a client can upload content 140 for a protocol 134, according to example embodiments. As further illustrated, second portion 902 may include sections 962-966 (continued from FIG. 9D and FIG. 9E above). Via section 962-966, a user may provide outline information for various portions of the update.

FIG. 9G is a continuation of graphical user interface 950 through which a client can upload content 140 for a protocol 134, according to example embodiments. As further illustrated, second portion 902 may include sections 968-972 (continued from FIGS. 9D-9F above). Via section 968-972, a user may provide outline information for various portions of the update.

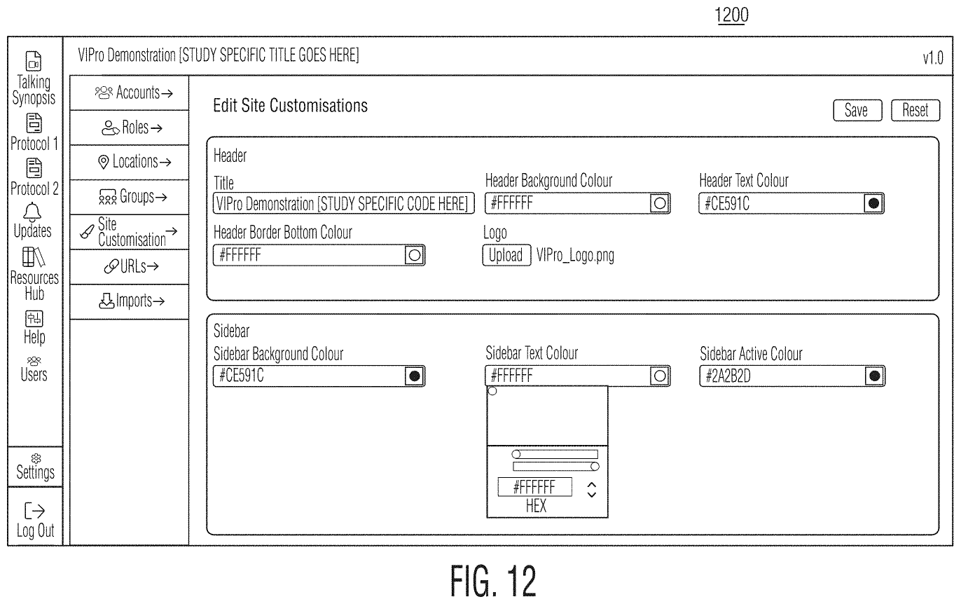

FIG. 12 illustrates an example view of a graphical user interface 1200 options to customize a website for a given protocol, according to example embodiments. Graphical user interface 1200 (hereinafter “GUI 1200”) may correspond to a GUI generated by interface agent 120. In some embodiments, GUI 1200 may be a web page presented in a web browser application (e.g., application 112) of user device 106 or application 110 of client device 102. In some embodiments, GUI 1200 may be a graphical user interface generated by a native application (e.g., application 112) executing on user device 106 or application 110 of client device 102.

As illustrated, GUI 1200 may allow a user to customize certain aspects of the website. For example, via GUI 1200 a user my edit header text, header background color, header text color, header border color, logos, sidebar colors, side bar text colors, side bar active colors, and the like.

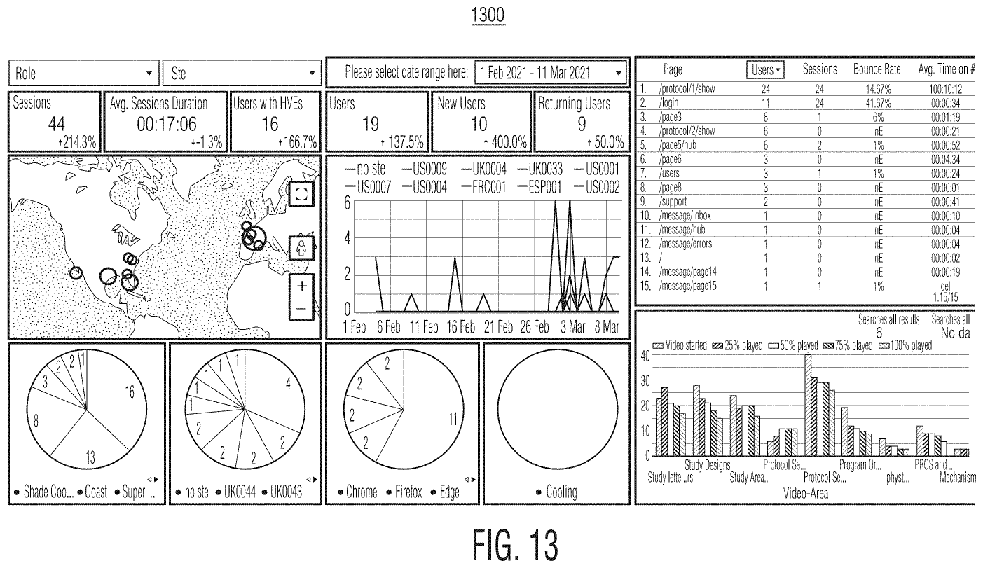

FIG. 13 illustrates an example view of a graphical user interface 1300 providing a metrics dashboard, according to example embodiments. Graphical user interface 1300 (hereinafter “GUI 1300”) may correspond to a GUI generated by interface agent 120. In some embodiments, GUI 1300 may be a web page presented in a web browser application (e.g., application 112) of user device 106 or application 110 of client device 102. In some embodiments, GUI 1300 may be a graphical user interface generated by a native application (e.g., application 112) executing on user device 106 or application 110 of client device 102.

As shown GUI 1300 may provide various metrics about a given protocol. For example, as shown, GUI 1300 may capture metrics such as, but not limited to, a geographic location of each user, a number of sessions, an average duration of sessions, a number of total users, a number of returning users, a number of returning users, and which pages of the protocol were viewed.

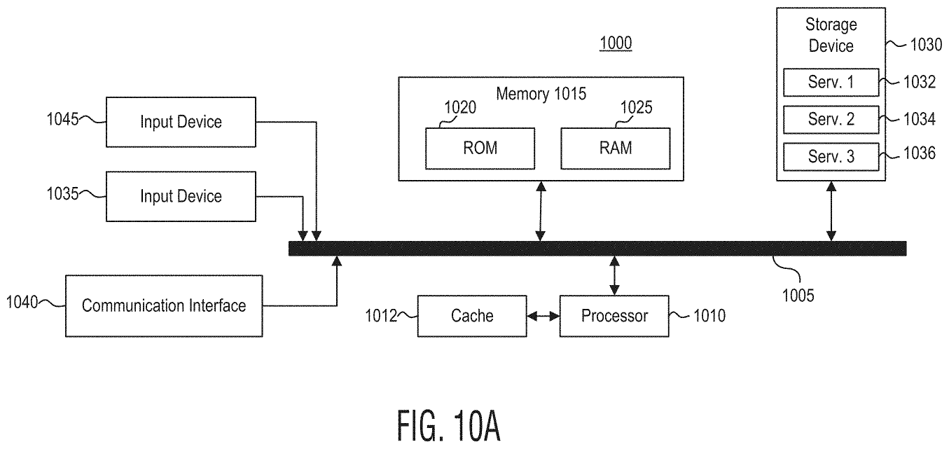

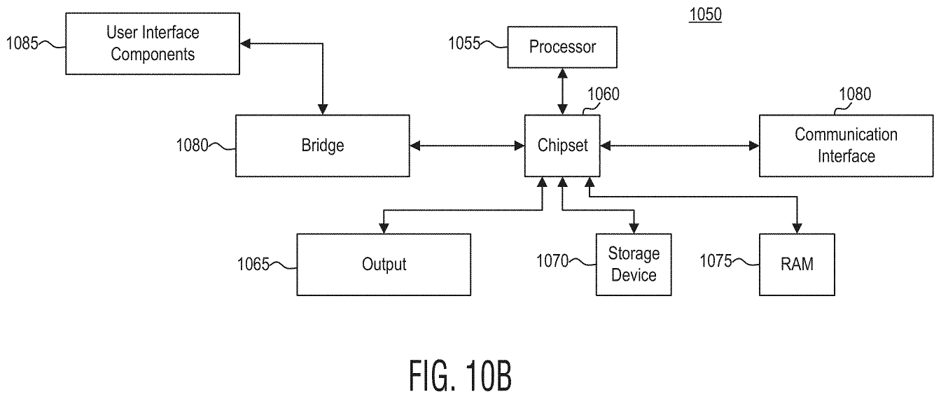

FIG. 10A illustrates a system bus computing system architecture 1000, according to example embodiments. One or more components of system 1000 may be in electrical communication with each other using a bus 1005. System 1000 may include a processor (e.g., one or more CPUs, GPUs or other types of processors) 1010 and a system bus 1005 that couples various system components including the system memory 1015, such as read only memory (ROM) 1020 and random access memory (RAM) 1025, to processor 1010. System 1000 can include a cache of high-speed memory connected directly with, in close proximity to, or integrated as part of processor 1010. System 1000 can copy data from memory 1015 and/or storage device 1030 to cache 1012 for quick access by processor 1010. In this way, cache 1012 may provide a performance boost that avoids processor 1010 delays while waiting for data. These and other modules can control or be configured to control processor 1010 to perform various actions. Other system memory 1015 may be available for use as well. Memory 1015 may include multiple different types of memory with different performance characteristics. Processor 1010 may be representative of a single processor or multiple processors. Processor 1010 can include one or more of a general purpose processor or a hardware module or software module, such as service 1 1032, service 2 1034, and service 3 1036 stored in storage device 1030, configured to control processor 1010, as well as a special-purpose processor where software instructions are incorporated into the actual processor design. Processor 1010 may essentially be a completely self-contained computing system, containing multiple cores or processors, a bus, memory controller, cache, etc. A multi-core processor may be symmetric or asymmetric.

To enable user interaction with the computing device 1000, an input device 1045 which can be any number of input mechanisms, such as a microphone for speech, a touch-sensitive screen for gesture or graphical input, keyboard, mouse, motion input, speech and so forth. An output device 1035 can also be one or more of a number of output mechanisms known to those of skill in the art. In some instances, multimodal systems can enable a user to provide multiple types of input to communicate with computing device 1000. Communications interface 1040 can generally govern and manage the user input and system output. There is no restriction on operating on any particular hardware arrangement and therefore the basic features here may easily be substituted for improved hardware or firmware arrangements as they are developed.

Storage device 1030 may be a non-volatile memory and can be a hard disk or other types of computer readable media that can store data that are accessible by a computer, such as magnetic cassettes, flash memory cards, solid state memory devices, digital versatile disks, cartridges, random access memories (RAMs) 1025, read only memory (ROM) 1020, and hybrids thereof.

Storage device 1030 can include services 1032, 1034, and 1036 for controlling the processor 1010. Other hardware or software modules are contemplated. Storage device 1030 can be connected to system bus 1005. In one aspect, a hardware module that performs a particular function can include the software component stored in a computer-readable medium in connection with the necessary hardware components, such as processor 1010, bus 1005, display 1035, and so forth, to carry out the function.

FIG. 10B illustrates a computer system 1050 having a chipset architecture that can be used in executing the described methods and generating and displaying a graphical user interface (GUI). Computer system 1050 may be an example of computer hardware, software, and firmware that can be used to implement the disclosed technology. System 1050 can include one or more processors 1055, representative of any number of physically and/or logically distinct resources capable of executing software, firmware, and hardware configured to perform identified computations. One or more processors 1055 can communicate with a chipset 1060 that can control input to and output from one or more processors 1055. In this example, chipset 1060 outputs information to output 1065, such as a display, and can read and write information to storage device 1070, which can include magnetic media, and solid state media, for example. Chipset 1060 can also read data from and write data to RAM 1075. A bridge 1080 for interfacing with a variety of user interface components 1085 can be provided for interfacing with chipset 1060. Such user interface components 1085 can include a keyboard, a microphone, touch detection and processing circuitry, a pointing device, such as a mouse, and so on. In general, inputs to system 1050 can come from any of a variety of sources, machine generated and/or human generated.

Chipset 1060 can also interface with one or more communication interfaces 1090 that can have different physical interfaces. Such communication interfaces can include interfaces for wired and wireless local area networks, for broadband wireless networks, as well as personal area networks. Some applications of the methods for generating, displaying, and using the GUI disclosed herein can include receiving ordered datasets over the physical interface or be generated by the machine itself by one or more processors 1055 analyzing data stored in storage 1070 or 1075. Further, the machine can receive inputs from a user through user interface components 1085 and execute appropriate functions, such as browsing functions by interpreting these inputs using one or more processors 1055.

It can be appreciated that example systems 1000 and 1050 can have more than one processor 1010 or be part of a group or cluster of computing devices networked together to provide greater processing capability.

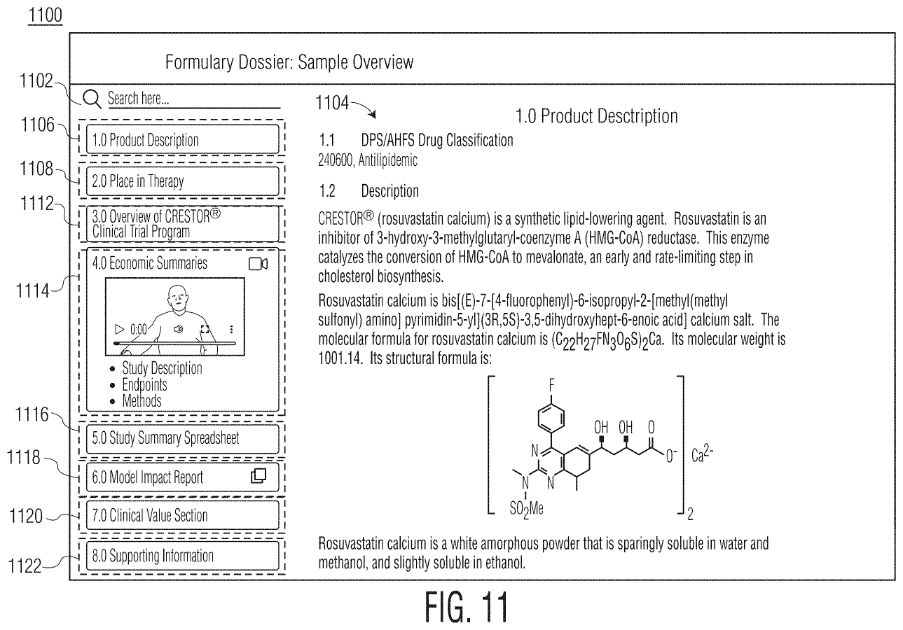

Although the above description includes operations directed to generating a portal for a clinical trial protocol, those skilled in the art recognize that the operations may be extended to delivery of other types of medical information. For example, the operations described herein may be extended to generating a portal for prescription information, drug registration dossiers, and the like. For example, FIG. 11 illustrates an example view of a graphical user interface 1100 through which a user can upload content for a formulary dossier, according to example embodiments. Graphical user interface 1100 (hereinafter “GUI 1100”) may correspond to a GUI generated by interface agent 120. In some embodiments, GUI 1100 may be a web page presented in a web browser application (e.g., application 110) of client device 102. In some embodiments, GUI 1100 may be a graphical user interface generated by a native software application (e.g., application 110) executing on client device 102.

As illustrated, GUI 1100 may correspond to a webpage associated with a given dossier. GUI 200 may include at least a first portion 1102 and a second portion 1104. First portion 1102 may correspond to a navigation pane of GUI 200. First portion 1102 may include one or more graphical elements 1108-1122 (e.g., buttons, links, etc.) selectable by the user to cause GUI 1100 (e.g., the browser or native application) to send a message to organization computing system 104 requesting information associated with each graphical element 1108-1122.

Second portion 1104 may include information associated with a selected graphical element 1108-1122. For example, as illustrated, second portion 1104 may include a product description responsive to a user selecting graphical element 1108 corresponding to “product description.”

While the foregoing is directed to embodiments described herein, other and further embodiments may be devised without departing from the basic scope thereof. For example, aspects of the present disclosure may be implemented in hardware or software or a combination of hardware and software. One embodiment described herein may be implemented as a program product for use with a computer system. The program(s) of the program product define functions of the embodiments (including the methods described herein) and can be contained on a variety of computer-readable storage media. Illustrative computer-readable storage media include, but are not limited to: (i) non-writable storage media (e.g., read-only memory (ROM) devices within a computer, such as CD-ROM disks readably by a CD-ROM drive, flash memory, ROM chips, or any type of solid-state non-volatile memory) on which information is permanently stored; and (ii) writable storage media (e.g., floppy disks within a diskette drive or hard-disk drive or any type of solid state random-access memory) on which alterable information is stored. Such computer-readable storage media, when carrying computer-readable instructions that direct the functions of the disclosed embodiments, are embodiments of the present disclosure.

It will be appreciated to those skilled in the art that the preceding examples are exemplary and not limiting. It is intended that all permutations, enhancements, equivalents, and improvements thereto are apparent to those skilled in the art upon a reading of the specification and a study of the drawings are included within the true spirit and scope of the present disclosure. It is therefore intended that the following appended claims include all such modifications, permutations, and equivalents as fall within the true spirit and scope of these teachings.

What is claimed:

1. A computer-implemented method for generating a protocol portal, comprising:
receiving, by a computing system, a request to generate a protocol portal from a client device;
generating, by the computing system, the protocol portal for the client device, the protocol portal comprising one or more protocol portal templates;
receiving, by the computing system, content for a protocol associated with the protocol portal from the client device;
transforming, by the computing system, the received content into a format compatible with the protocol portal;
dynamically tagging, by the computing system, the content uploaded by the client device; and
generating, by the computing system, one or more graphical user interfaces for the protocol portal using the received content and the protocol portal templates.

2. The computer-implemented method of claim 1, wherein transforming, by the computing system, the received content into a format compatible with the protocol portal comprises:
converting the received content into a searchable format.

3. The computer-implemented method of claim 1, wherein the content comprises one or more of video content, audio content, image content, and text content.

4. The computer-implemented method of claim 1, further comprising:
receiving, by the computing system from a user device, a search request comprising a search term via the one or more graphical user interfaces for the protocol portal.

5. The computer-implemented method of claim 4, further comprising:
comparing, by the computing system, the search term in the search request against text content of the protocol to identify search results.

6. The computer-implemented method of claim 4, further comprising:
comparing, by the computing system, the search term in the search request against tags associated with content to identify search results.

7. The computer-implemented method of claim 1, further comprising:
monitoring, by the computing system, user compliance throughout protocol training.

8. A non-transitory computer-readable medium including one or more instructions that, when executed by a computing system, cause the computing system to perform operations comprising:
receiving, by the computing system, a request to generate a protocol portal from a client device;
generating, by the computing system, the protocol portal for the client device, the protocol portal comprising one or more protocol portal templates;
receiving, by the computing system, content for a protocol associated with the protocol portal from the client device;
transforming, by the computing system, the received content into a format compatible with the protocol portal;
dynamically tagging, by the computing system, the content uploaded by the client device; and
generating, by the computing system, one or more graphical user interfaces for the protocol portal using the received content and the protocol portal templates.

9. The non-transitory computer-readable medium of claim 8, wherein transforming, by the computing system, the received content into a format compatible with the protocol portal comprises:
converting the received content into a searchable format.

10. The non-transitory computer-readable medium of claim 8, wherein the content comprises one or more of video content, audio content, image content, and text content.

11. The non-transitory computer-readable medium of claim 8, wherein the operations further comprise:
receiving, by the computing system from a user device, a search request comprising a search term via the one or more graphical user interfaces for the protocol portal.

12. The non-transitory computer-readable medium of claim 11, wherein the operations further comprise:
comparing, by the computing system, the search term in the search request against text content of the protocol to identify search results.

13. The non-transitory computer-readable medium of claim 11, wherein the operations further comprise:
comparing, by the computing system, the search term in the search request against tags associated with content to identify search results.

14. The non-transitory computer-readable medium of claim 8, wherein the operations further comprise:
monitoring, by the computing system, user compliance throughout protocol training.

15. A system, comprising:
a processor; and
a memory having programming instructions stored thereon, which, when executed by the processor, causes the system to perform operations comprising:
receiving a request to generate a protocol portal from a client device;
generating a protocol portal for the client device, the protocol portal comprising one or more protocol portal templates;
receiving content for a protocol associated with the protocol portal from the client device;
transforming the received content into a format compatible with the protocol portal;
dynamically tagging the content uploaded by the client device; and
generating one or more graphical user interfaces for the protocol portal using the received content and the protocol portal templates.

16. The system of claim 15, wherein transforming the received content into a format compatible with the protocol portal comprises:
converting the received content into a searchable format.

17. The system of claim 15, wherein the content comprises one or more of video content, audio content, image content, and text content.

18. The system of claim 15, wherein the operations further comprise:
receiving, from a user device, a search request comprising a search term via the one or more graphical user interfaces for the protocol portal.

19. The system of claim 18, wherein the operations further comprise:
comparing the search term in the search request against text content of the protocol to identify search results.

20. The system of claim 18, wherein the operations further comprise:
comparing the search term in the search request against tags associated with content to identify search results.
